(19) United States
(12) Patent Application Publication
(10) Pub. No.: US 2024/0277984 A1
(43) Pub. Date: Aug. 22, 2024

(54) Hydrocephalus shunt valve

(71) Applicant: Liverpool John Moores University, Liverpool (GB)

(72) Inventors: Marco Messina, Liverpool (GB); Osman Salih, Liverpool (GB); Dhiya Al-Jumeily, Liverpool (GB)

(21) Appl. No.: 18/172,687

(22) Filed: Feb. 22, 2023

(51) Int. Cl. A61M 27/00

(52) U.S. Cl. CPC A61M 27/006; A61M 2205/0294; A61M 2205/3344

(57) ABSTRACT

A valve for a shunt useable in treatment of hydrocephalus is described. The valve comprises an inlet in fluid communication with an outlet, a valve closure element, a mechanical biasing element configured to bias the valve closure element towards the inlet, and an electromechanical actuator configured to manipulate the mechanical biasing element. A biasing force applied to the valve closure element by the mechanical biasing element determines an operating pressure of the valve, and manipulating the mechanical biasing element by the electromechanical actuator allows for adjustment of the valve operating pressure by suitably varying the biasing force.

### FIELD OF THE INVENTION

The present disclosure relates to a hydrocephalus shunt valve; that is, a valve apparatus for a shunt which is surgically implantable to treat hydrocephalus. In particular, the present disclosure is concerned with a mechatronic valve which combines the advantageously passive nature of classical shunt valves with the controllability of fully automated valves.

### BACKGROUND

Hydrocephalus is a disorder caused by dynamic imbalance between the production and absorption of cerebrospinal fluid (CSF). It is typically managed using a device called a shunt, which drains excess CSF. A shunt is formed from three main components: (1) an inflow (proximal catheter) which drains CSF from lateral ventricles in the brain; (2) a valve mechanism which regulates intracranial pressure by controlling fluid flow through the shunt; and (3) an outflow (distal catheter) which directs CSF from the valve to a drainage site such as the abdominal cavity or heart.

Currently there are two types of valves. Fixed differential pressure valves (DPV) and Programmable (adjustable) valves. Both valves operation is based on the pressure difference between the intracranial pressure (ICP) and the drainage site pressure. With adjustable valves there is the option of adjusting opening pressure externally using magnetic devices, but once the adjustable valve's opening pressure is set, they behave exactly like DPV shunts.

There are several drawbacks of the currently used shunting systems: (i) it does not take into consideration the changes in the dynamic behaviour of ICP which vary, not only from one patient to another but also for the same patient depending on age, health, and other elements; (ii) current shunting system tends to encourage the patient shunt dependency to increase with time due to the lack of personalization; (iii) the system lacks proactivity and does not recognize the rise in ICP due to normal events such as coughing, sneezing, and even switching between standing and prone positions, which leads to unnecessary drainage (i.e. over-drainage)—in such a scenario the drained CSF could take hours to be re-produced again; (iv) any shunt malfunctions cannot be detected until they manifest clinically as symptoms, which can be life-threatening based on the type of malfunction and the patient's condition. As a result, the monitoring and follow-up of shunted patients is an essential part of ongoing patient safety. Current statistics indicate that 30%-40% of shunts fail within the first year of use, 50% fail within the first two years, and 90% of them fail after five years. According to a UK study, there were 3000 shunt operations in the UK in 2017, 1660 for paediatric patients and 1400 for adults: of these, 66.5% of paediatric operations 47% of adult's operations were for shunt revision as appose to primary first-time installation.

A recent trend has been the development and use of silicon membranes in place of a traditional valve, however while offering some advantages (pressure-sensitivity, compactness), these designs still have the same fundamental problems noted above.

Hence an improved shunt valve which improves upon previously available designs is highly desirable.

### SUMMARY

The example embodiments have been provided with a view to addressing at least some of the difficulties that are encountered with current shunts and shunt valves, whether those difficulties have been specifically mentioned above or will otherwise be appreciated from the discussion herein.

In particular, it is an aim of the present disclosure to provide a solution which is compatible with the idea of a “smart shunt”, which is a shunt device which is controllable based on one or more sensory inputs. The present disclosure is directed towards a mechatronic shunt valve which may form a core component of a smart shunt.

The present invention is defined according to the independent claims. Additional features will be appreciated from the dependent claims and the description herein. Any embodiments which are described but which do not fall within the scope of the claims are to be interpreted merely as examples useful for a better understanding of the invention.

Suitably, in one aspect of the invention there may be provided a valve apparatus/component of a shunt which is useable in the treatment of hydrocephalus. The valve apparatus comprises an inlet in fluid communication with an outlet, a valve closure element and a mechanical biasing element configured to bias the valve closure element towards the inlet. A biasing force applied to the valve closure element by the mechanical biasing element determines an operating pressure of the valve—that is a minimum fluid pressure able to flow into the valve through the inlet. The valve apparatus also comprises an electromechanical actuator configured to manipulate the mechanical biasing element to adjust the biasing force to vary the operating pressure of the valve. Suitably, the valve apparatus (when suitably in use in a surgically implanted hydrocephalus shunt) may control an ICP at which CSF is able to drain away from the cranium.

In some examples the electromechanical actuator comprises a stator driven by an ultrasonic motor, which may preferably include a piezoelectric element. In a further preferred example, the piezoelectric element comprises piezoceramic material, such as PTZ-5H. In some examples the ultrasonic motor may operate at voltage between 3 and 5 volts, inclusive.

Suitably in some examples the electromechanical actuator is self-locking, so that once an operating pressure has been set by the electromechanical actuator the valve remains operating at that pressure without continued electrical current or voltage being applied.

In some examples the ultrasonic motor comprises a protrusion configured to grip the stator. The protrusion may be arranged on a first surface of the piezoelectric element orthogonal to a second surface of the piezoelectric element to which voltage is applied. In this way may the mechanical biasing element be actuated by ultrasonic motor. In particular, the ultrasonic motor may apply a step-wise manipulation which comprises a repeated series of gripping, moving, and releasing the stator by the protrusion. Suitably the protrusion may be arranged to straddle a boundary between a first electrode of the piezoelectric element and a second electrode of the piezoelectric element.

In some examples the mechanical biasing element comprises a substantially linear spring, and may be formed from a material comprising Magnesium AZ91E-T6.

In some examples the valve closure element may be substantially ball shaped, preferably having a diameter of at least 1.3 mm, particularly when the inlet is arranged with a diameter of substantially 1 millimetre ‘mm’. In some examples the valve further comprises a valve seat configured to receive the valve closure element—that is, the valve closure element may be configured to rest in the valve seat—and which may comprise a shape suitably configured to correspond to a shape of the valve closure element (e.g., a socket to substantially match a ball shaped closure element).

In a related aspect of the invention there may be provided a shunt for use in treatment of hydrocephalus. The shunt may comprise the aforementioned shunt valve and any of its corresponding features.

In a further related aspect of the invention there may be provided a method of treatment of hydrocephalus comprising surgically implanting the aforementioned shunt including the aforementioned valve apparatus. Suitably, in some examples, the method may comprise adjusting the operating pressure of the electromechanical actuator post-surgery by e.g., arranging for exposed/accessible electrical connectors to the valve apparatus as part of the initial surgery. In this way the valve operating pressure may be suitably adjusted without a need for further surgery at a later date.

### BRIEF DESCRIPTION OF THE DRAWINGS

For a better understanding of the present disclosure reference will now be made by way of example only to the accompanying drawings, in which:

FIG. 1 shows a cut through an example improved valve apparatus for a hydrocephalus shunt;

FIG. 2 shows an example shunt surgically implanted to treat hydrocephalus;

FIG. 3 shows boundary conditions for a computation fluid dynamic model of an example valve apparatus;

FIG. 4 shows an example fluid velocity profile around an inlet of an example valve apparatus;

FIG. 5 shows an example ultrasonic motor element;

FIG. 6 shows example dimensions for an example ultrasonic motor element;

FIG. 7 shows deformation at resonant frequencies for various sheets of piezoceramic material used in the ultrasonic element;

FIG. 8 shows relative displacement on the top plane of an example piezoelectric element;

FIG. 9 shows computational modelling of an example ultrasonic motor element;

FIG. 10 shows motion of an example ultrasonic motor (particular a gripper part) when a sin-wave voltage is applied; and

FIG. 11 shows a graph of X-Y displacement of the gripper part of the ultrasonic motor when the sin-wave voltage is applied.

### DETAILED DESCRIPTION

At least some of the following example embodiments provide an improved hydrocephalus shunt valve. The example device is simple to operate and convenient to adjust as necessary for treatment of a patient, without revision surgery. Many other advantages and improvements will be appreciated from the discussion herein.

In the foregoing, there are three main considerations which have been gone into the design of the presently disclosed shunt valve. The first consideration is size and compactness: it is preferably that the layout and components are arranged as efficiently as possible, in as small a space as possible, as the valve will be ultimately implanted under the skin, typically in the area above the ear. Suitably, it is preferably to design the valve in a way that enables all components to be arranged in a single housing for compactness. The second consideration is component suitability, which relates to the chosen materials having biological and magnetic resonance imaging compatibility. Other issues are related to patient inconvenience such as noise (i.e. from friction) as the device is used in the patient's head. The third consideration is power consumption, as the valve is meant to be a long-term management system. Therefore, it is desirable to keep power draw at a minimum. It will be appreciated from the discussion herein how each of the described features aligns with one or more of these design goals.

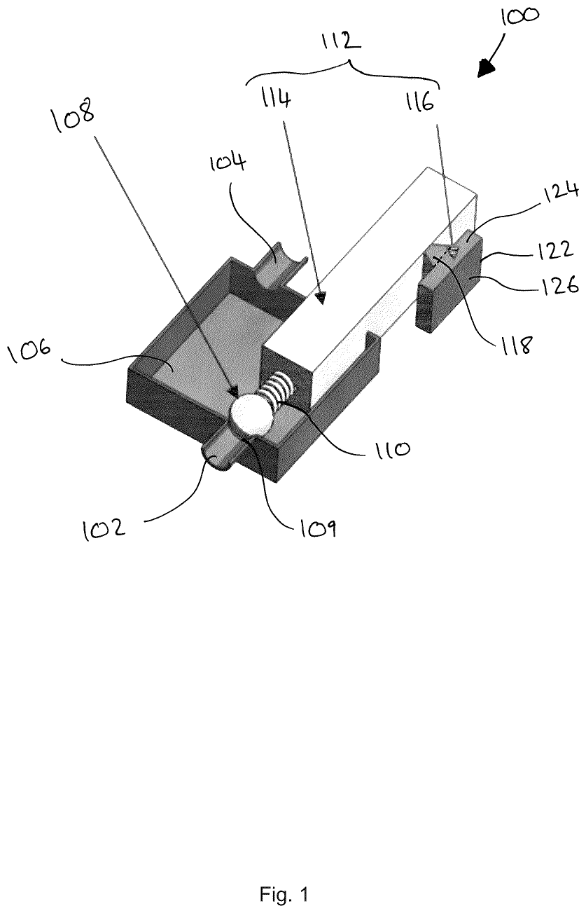

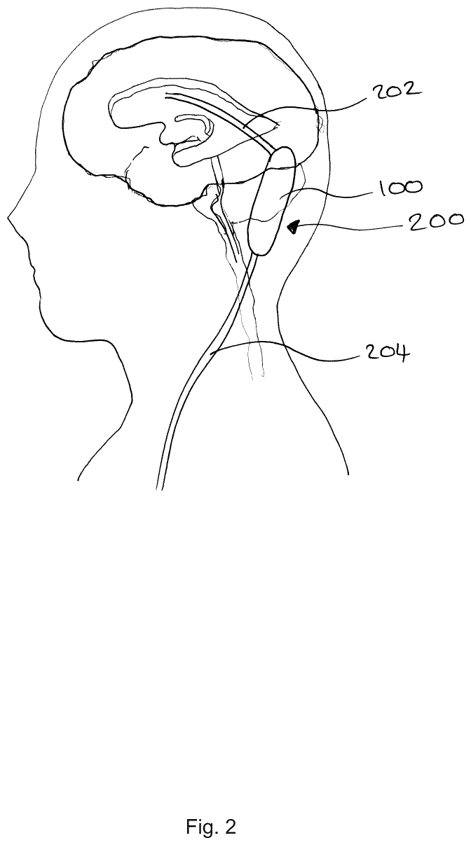

FIG. 1 shows an example improved hydrocephalus shunt valve 100 which may form a valve component for a surgically implantable shunt 200 for use in the treatment of hydrocephalus, as shown in FIG. 2. Suitably, the hydrocephalus shunt valve 100 is designed to regulate fluid flow through the shunt 200.

Suitably, the shunt valve 100 comprises an inlet 102 which is configured to couple to a proximal catheter 202 of the shunt 200, and an outlet 104 configured to couple to a distal catheter 204 of the shunt 200. The inlet 102 and outlet 104 are arranged in fluid communication via a fluid chamber 106. Suitably, consistent with current devices, the inlet 102 and outlet 104 may each have an opening (i.e., diameter) in the range of 0.5 millimetre (mm) to 1.5 mm, preferably substantially 1 mm. The fluid chamber 106 may be designed in a substantially square configuration with side length approximately 6 mm.

The shunt valve 100 comprises a closure element 108 configured to close the inlet 102. That is, the closure element 108 is configured to block a fluid flow into the fluid chamber 106 through the inlet 102. Suitably, the closure element 108 may be configured to abut the edge of the inlet 102. Or put another way, the closure element 108 may comprise a surface with a size greater than the diameter of the inlet: for example, if the diameter of the inlet is 1 mm, then the size of the closure element 108, or rather a part thereof which abuts the inlet, may be greater than 1 mm.

In the present example the closure element 108 is a ball; suitably the diameter of the ball 108 is greater than the diameter of the inlet 102. In particular, where the diameter of the inlet 102 is substantially 1 mm, the diameter of the ball may be in the range of 1.1 mm to 2 mm, preferably 1.3 mm. In other examples, not shown the closure element may be differently shaped, such as being cone shaped, or disc shaped, with suitable considerations for the size of the shapes in relation to the inlet 102.

The valve 100 also comprises a mechanical biasing element 110 configured to bias the valve closure element towards the inlet 102; that is, to bias the closure element 108 to close the inlet 102 by ensuring contact against the inlet boundary. As will be explored in further detail below, the biasing force applied by the mechanical biasing element 110 determines a minimum threshold fluid pressure (i.e., a minimum intercranial fluid pressure) which can force open the valve closure element 108. Here, opening the closure element 108 means moving it away from the surface/edge/boundary of the inlet 102 to allow fluid to flow through the inlet 102 into the fluid chamber 106. In other words, the closure element 108 and mechanical biasing element 110 work in combination to act as a pressure operated switch which is open above a set threshold fluid pressure and closed below the threshold fluid pressure. Suitably it may be considered that the biasing force applied by the mechanical biasing element 110 sets an operating pressure of the valve 100.

Preferably, the mechanical biasing element 110 comprises a linear spring, more specifically an extension spring, to provide the desired biasing towards the inlet 102. Preferably the spring is formed of Magnesium AZ91E-T6, due to its suitability for use in the human body and safety of use under magnetic resonance imaging (MRI). In a preferred example the linear spring has a free length of approximately 4.22 mm, a wire diameter of 0.06 mm, and an outer diameter of 1.8 mm. More generally, the spring is suitably dimensioned to reside within the chamber 106 while allowing for displacement. Suitably the spring may comprise a free length between 1 mm and 5 mm, an outer diameter between 1 mm and 3 mm, and a wire diameter between 0.01 mm and 1 mm.

In some examples the valve comprises a valve seat 109 located within the fluid chamber 106 proximate to the inlet 102. Suitably, the closure element 108 may be configured to rest in the valve seat 109, and so suitably the valve seat 109 is configured to substantially correspond to the shape of the valve closure element 108. Suitably, where the closure element 108 is a ball, the valve seat 109 is a concave socket. The socket 112 may be configured to allow slippage of the ball 108 (e.g., by having a slightly different radius of curvature), so that the fluid pressure from the inlet 102 can move the ball 108 within the socket 109 (away from the inlet 102) when the fluid pressure is above the desired threshold, but to not allow the ball 108 out of the socket 109. It will be appreciated that FIG. 1 shows a cut through the middle of the inlet 102, outlet 104, and chamber 106, and so suitably in some examples (not shown) the socket 109 may indeed be two sockets arranged on opposite (inner) surfaces of the fluid chamber 106, to further aid in preventing the ball 108 from leaving the socket 109, and so ensure operation of the device no matter its orientation.

It will be appreciated that the valve seat 109 may take various forms as appropriate for exact type of closure element used. For example, instead of a socket, the valve seat 109 may be in the form of a channel which restricts motion of the closure element to be orthogonal to the opening of the inlet (but not lateral to it). Such a form may be particularly appropriate where the closure element 108 is a cone or a disc.

The valve 100 also comprises an electromechanical actuator 112 coupled to the mechanical biasing element 110 (and by extension, the closure element 108). The actuator 112 is configured to set the threshold fluid pressure which can open the closure element 108 by manipulating the mechanical biasing element 110 (e.g., when the biasing element 110 is a spring, by deforming the spring). Suitably, deforming (or more generally, manipulating) the mechanical biasing element 110 adjusts the biasing force applied to the closure element 108, thereby adjusting the fluid pressure required to force open the inlet 102 to allow fluid to flow into the chamber 106 and through the shunt 200. In this way the operating pressure of the valve 100 may be suitably varied so that the valve 100 regulates fluid flow through the shunt device 200; i.e., by setting a desired fluid pressure that is required before fluid is allowed to drain away through the shunt 200.

In a preferred example, as shown, the electromechanical actuator 112 comprises a stator 114 driven by an ultrasonic motor 116. Suitably, the ultrasonic motor 116 comprises a gripper 118 by which the ultrasonic motor engages the stator 114 on a surface of the stator 120 running substantially parallel to the direction of biasing by the mechanical biasing element 110.

Movement of the gripper 118 part of the ultrasonic motor 116 (specifically, a rotational movement in a plane orthogonal to the surface 120) may cause the stator 114 to move due to friction—i.e., by applying a net directional force on the stator 114 either towards or away from the inlet 102. Put another way, rotational movement of the gripper 118 is converted to linear movement of the stator 114. In this way the ultrasonic motor 116 causes the stator 114 to deform the mechanical biasing element to change the threshold pressure at which fluid is allowed through the inlet 102 into the chamber 106. The gripper 118 may maintain contact with the stator 114 after it has moved the stator 114, so that the electromechanical actuator 112 is self-locking; that is, when the gripper 118 is no longer rotating, the gripper 118 holds the stator 114 in the position it was just moved to.

Although the preferred example shows only a single gripping element 118 in the form of a protrusion, it will be appreciated that more than one gripping element may be provided and such elements may also take different form factors.

Preferably the ultrasonic motor comprises a piezoelectric element 122. The gripper 118 is preferably arranged on a first (minor) surface 124 of the piezoelectric element 122 which is orthogonal to a second (major) surface 126 of the piezoelectric element 122. The second surface of the piezoelectric element 122 may be a surface to which voltage is applied. More specifically the gripper 118 may be arranged to straddle a boundary between a first electrode 128 and a second electrode 130 of the piezoelectric element 122. The first electrode 128 may be set to ground and the second electrode 130 may have a positive or negative voltage applied to it.

In the present example the gripper 118 preferably comprises aluminium oxide as it is a material with a high coefficient of friction. The piezoelectric element 122 may suitably comprise a piezoceramic material, such as PTZ-5H, which has better piezoelectric properties compared to other piezoceramics. The ultrasonic motor 116 may be configured to operate at voltage between 3 and 5 volts, inclusive, so that it may be powered by an implantable battery (i.e., a battery implanted alongside the shunt 200) while having long operational life.

### Computation Fluid Dynamic Modelling

Fluid flow through the shunt valve 100 may be suitably understood by the Navier-Stokes equations:

$$\frac{\partial ρ}{\partial t} + \frac{\partial ρ {u}_{i}}{\partial {x}_{i}} = 0 \qquad eq . 1$$

$$\frac{\partial ρ {u}_{i}}{\partial t} + \frac{\partial ρ {u}_{i} {u}_{j}}{\partial {x}_{j}} = - \frac{\partial P}{\partial {x}_{i}} + \frac{\partial ( μ \frac{\partial {u}_{i}}{\partial {x}_{j}} )}{\partial {x}_{j}} + {S}_{{M}_{i}} \qquad eq . 2$$

Equations eq. 1 & eq. 2 are known as the Continuity equation and the Momentum equation respectively. Where u is the Dynamic viscosity and SM; is a term that represent external effects.

CSF is extremely similar to water and thus properties of water may be suitably used in the simulation. A useful parameter is Reynold's number, which is calculated from:

$$R e = \frac{ρ VD}{μ} , \qquad eq . 3$$

where V is the velocity and D is the catheter diameter (i.e., the diameter of the inlet and outlet of the valve 100). For V, a typical CSF volume production in hydrocephalus patients may be taken to be around 2000 ml/day. D was taken to be 1 mm, to the average of inside diameters of proximal and distal catheters. For a shunt valve 100 arranged as above, the resulting Reynold's number indicates a laminar flow (Re around 36). This means that flow through the device cannot be regarded as a creeping flow (Re<<1), which would have meant that inertial effects could have been ignored. This is the similar to existing shunt devices, indicating why it is desirable to design shunt valves, like the present valve, which can cope with changes in inertia (e.g., when transitioning from prone to standing and vice versa) as this changes the hydrostatic pressure.

The shunt valve 100 is preferably designed to operate within a pressure range of 10 mmHg to 20 mmHg, corresponding to the typical range of ICP observed in hydrocephalus sufferers. That is, the mechanical biasing element 110 is configured to apply a biasing force to the closure element 108 equivalent to a fluid pressure of between 10 mmHg to 20 mmHg. Suitably, the electromechanical actuator 112 is arranged to control the mechanical biasing element 110 to set a specific pressure within that range, thereby setting the ICP above which the shunt valve is opened.

In a further preferred example, the shunt valve 100 is arranged to provide 11 discrete ICP points from 10 mmHg to 20 mmHg with 1 mmHg intervals. That is, the electromechanical actuator 112 is suitably controllable to adapt the mechanical biasing element 110 to change the opening pressure of the closure element in 1 mmHg intervals. Although it will be appreciated that other intervals could be utilised, if desirable, and indeed intervals which represent essentially a continuity between the upper and lower pressure design parameters.

It is desirable to take into consideration the effects of hydrostatic pressure. As a result, simulations were run for the 11 points under both default conditions and conditions where the hydrostatic pressure is in effect; here default conditions mean that the patient is supine, and under hydrostatic effect is when the patient is standing.

The simulation is based on pressure at the inlet point of the valve, and accordingly it is useful to consider pressure losses due to friction in the proximal catheter. The equations are as follows:

$${P}_{f r i c t i o n} = \frac{128 μ L Q}{π {D}^{4}} \qquad eq . 4$$

$${P}_{h} = ρ g h sin ( Ψ ) \qquad eq . 5$$

$${P}_{i n l e t} = I C P - {P}_{f r i c t i o n} \qquad eq . 6$$

$${P}_{h - i n l e t} = {P}_{f r i c t i o n} + {P}_{h} \qquad eq . 7$$

Here P_{friction} is pressure loss due to friction, Pₕ is the hydrostatic pressure, Pᵢₙₗₑₜ is the pressure at the valve inlet, and Pₕ₋ᵢₙₗₑₜ is the pressure at valve inlet in the standing position. L is the proximal catheter length, g is gravity acceleration, h is the column of fluid elevation, and Y is the angle of that column. Since the shunt 200 is placed near the ear to the back of the head, the length between the shunt 200 and the proximal catheter inlet 202 is h; based on intended deployment of the shunt 200, this can be approximated as 30 cm. The hydrostatic pressure is at its highest at the upright position and thus Y may be put as 90 degrees. Suitably the model accounts for 22 total pressure design points as each of the 11 discrete pressure points has two conditions, the default condition and the under hydrostatic pressure condition.

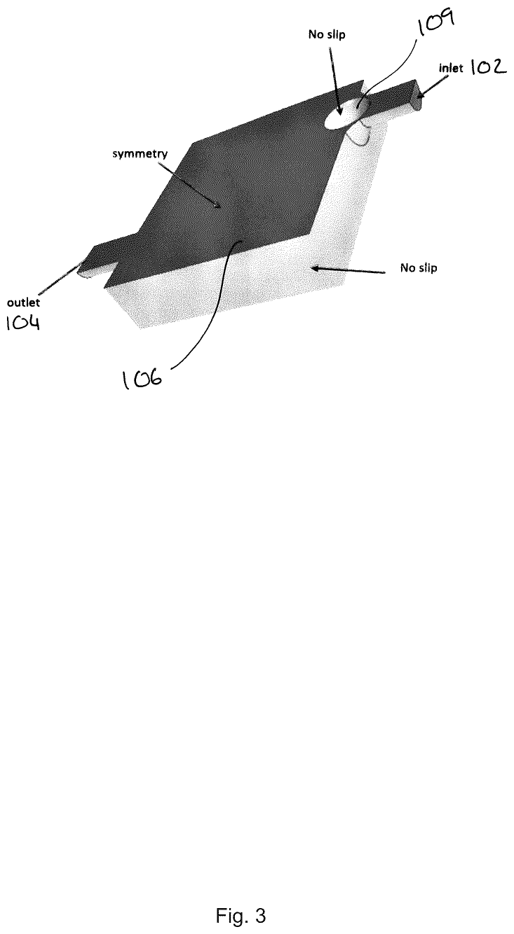

FIG. 3 shows the boundary conditions for a computation fluid dynamic model of the shunt valve 100 utilising the equations above. All walls including the closure element 108 (ball) are set to no-slip—zero velocity relative to the boundary—boundary conditions. The inlet 102 boundary condition is inlet pressure for each of the 22 pressure design points. The outlet 104 boundary pressure is determined by the intended usage of the shunt 200. For example, if the distal catheter is intended to drain the CSF into the heart's right atrium, then the outlet pressure may be estimated as approximately 6 mmHg.

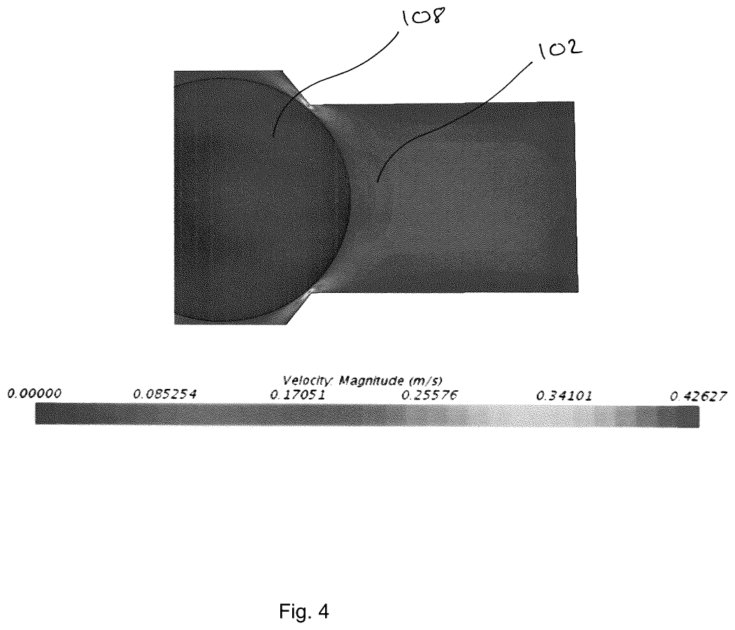

The model setup according to the above may be used to determine the velocity profile for the shunt valve 100. For example, FIG. 4 shows the velocity profile around the inlet at the minimum pressure point of 10 mmHg. High-velocity values are consternated in the area near the closure element 108 on the inlet 102 side. Force on the closure element 108 as a result of both fluid pressure and shear force may be extracted for each simulation. These force values are useful for designing the mechanical biasing element 110 (a spring in the present example) that controls the closure element 108. It has been determined that the preferred example of the valve 100 presently described offers outflow drainage between 42-300 mL/h (corresponding to the min to max pressure points). In terms of force on the closure element 108, this can be determined to be between 1.5-14.7×10⁻⁴ N. This includes the pressure points in the supine and standing position.

### Spring Design

The mechanical biasing element 110, which is a linear spring in the preferred example, may be suitably designed by an iterative process. Two models may be built corresponding to maximum displacement of the biasing element 110 at maximum force of 20 mmhg, and to minimum displacement of the biasing element 110 at minimum force of 10 mmHg. The boundary conditions used are a fixed end surface of one side of the mechanical biasing element 110 and applying displacement on the other end surface; more specifically, an end of the biasing element 110 coupled to the electromechanical actuator 112 is considered fixed, while an opposite end of the biasing element 110 attached to the closure element 108 is considered displaceable. For a spring with preferred parameters—i.e., free length, wire diameter, and outer diameter of the model are 4.22, 0.06, and 1.8 mm respectively—then maximum loading conditions at 20 mmHg correspond to a displacement of 2.22 mm while minimum loading conditions at 10 mmHg correspond to a displacement of the spring of 0.22 mm.

### Ultrasonic Motor Design

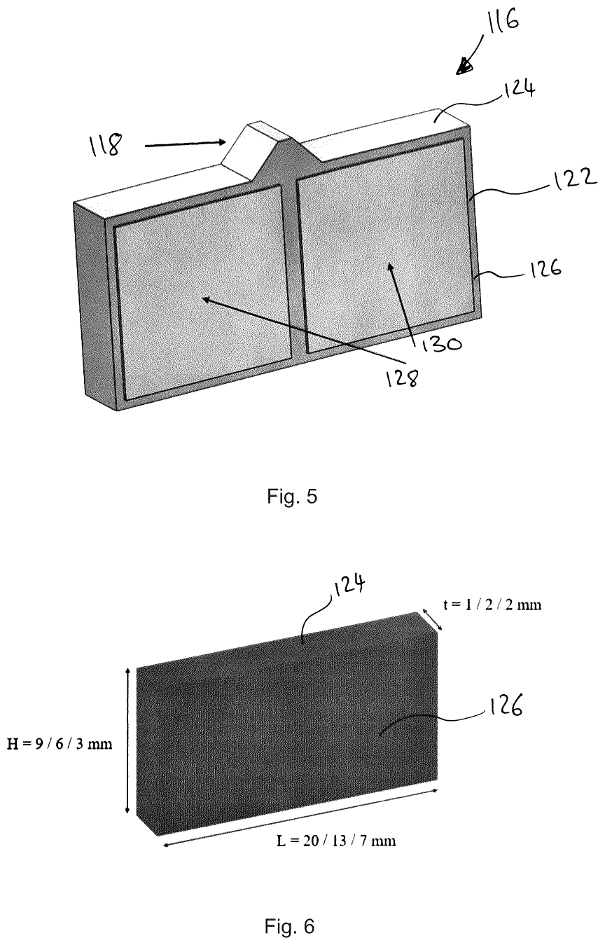

FIG. 5 shows the example electromechanical actuator 112 in more detail. The operation of the ultrasonic motor 116 depends on mechanical resonance. The ultrasonic motor 116 is excited using a high-frequency current. With the right frequency excitation, the motor 116 will be under resonance. The resulted deformation is then harvested through friction to induce displacement to the stator 114. The motor 116 is asymmetrically excited by applying a voltage through one of the electrodes 128, 130. This results in deformation that gives motion—utilizing tip 118 and stator 114 friction—in one direction. The main design process for the present motor 116 is to conduct a finite element analysis on the motor 116. A modal analysis is conducted to understand and visualize different modes of excitation and which one of these modes is to be used. This is considered a frequency extraction step. The other step is to simulate partial excitement of the element by applying a voltage at the extracted frequency.

Acoustic waves spread as compression waves in solid bodies. In small enough bodies this can result in creating standing waves (two waves with the same amplitude and frequency moving in opposite directions). However, in bodies with two dimensions of the same order such as metal sheets, two-dimensional standing waves are observed. These modes types are termed E(K,I) where E refers to “extensional” as these modes cause planar extension. K and I are the number of half wavelengths in the X & Y direction respectively. For a piezoceramic plate with a length (L) in the X direction, height (H) of 0.5 L in the Y direction, and polarization in the thickness (t) in the Z direction two E(3,1) modes are observed. These modes consist of 2D standing waves. The one on the X-direction has 3 half wavelengths (L=3λₓ/2), and the one on the Y direction has a one-half wavelength (H=λ_{y}/2). The mode shape can be described as:

$${U}_{x} ( x , y , t ) = A sin - ( \frac{3 π}{L} x ) ( cos ( \frac{π}{H} y ) - 1 ) sin ( ω t ) \qquad eq . 8$$

$${U}_{y} ( x , y , t ) = B ( 1 \pm C cos ( \frac{3 π}{L} x ) ) sin ( \frac{π}{H} y ) sin ( ω t ) \qquad eq . 9$$

Where Uₓ & U_{y} are displacements in the X & Y directions. A, B, C are geometrical and material functions. A modal analysis may be conducted to find the resonance frequency of three piezo elements. In the present example the dimensions are 7×3×1, 13×6×2, and 20×7×2 mm as shown in FIG. 6.

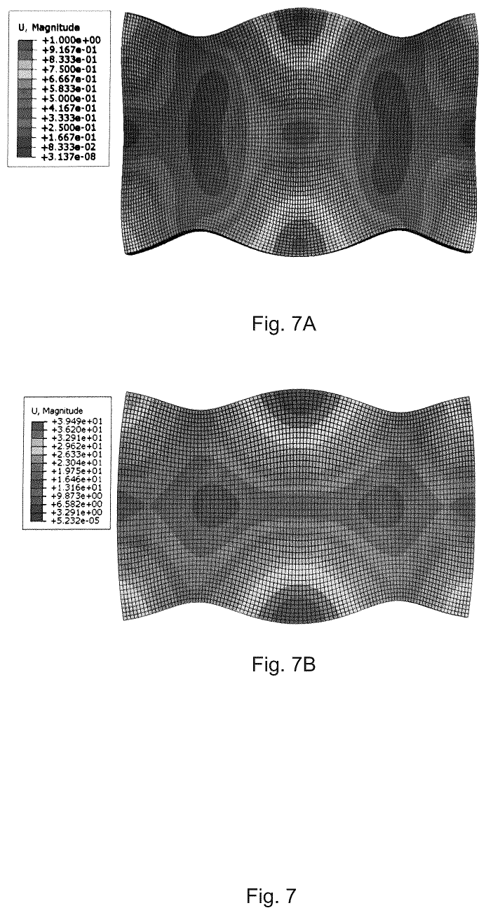

FIG. 7 shows deformation at the resonant frequency for (A) a 13×6×2 mm sheet of piezoceramic and (B) a 20×7×2 mm sheet of piezoceramic. The first E(3,1) mode of the 13×6×2 mm and the 20×7×2 mm sheet occurs at 294.8 kHz and 178 KHz respectively. As can be seen, the deformation in this cases is generally symmetric in both X & Y axis, which may be achieved by 2 pairs of standing waves.

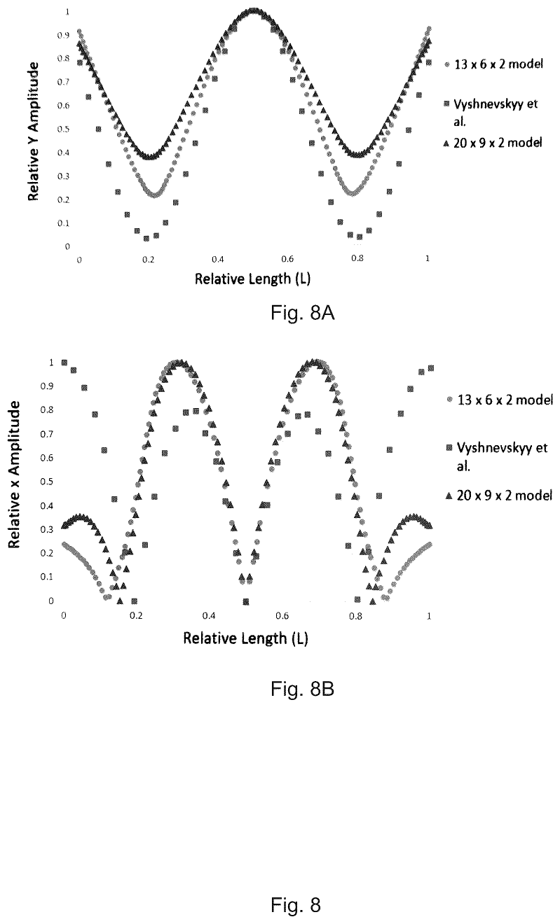

FIG. 8 shows relative displacement on the top plane of an example piezoelectric element 122 at t/2 (i.e., in the middle of the piezoceramic sheet). The Y relative displacement (amplitude) is compared with that of Vyshnevskyy et al. (Vyshnevskyy, Kovalev and Wischnewskiy, 2005). For Y amplitude, note that although the two sets of points—FIG. 8A—are not an exact match, their general profiles are the same. This is the same case when it comes to the X amplitude—Error! Reference source not found. FIG. 8B—where the two sets of points also do not exactly match but the same general profile.

The next step after confirming the E(3,1) mode frequency is to simulate

The operation of the ultrasonic motor 116 (in an E(3,1) mode) may be suitably simulated through partial excitation. A high-frequency current must be simulated through the piezo element 122 to understand what type of reaction is to be expected. In the following, a 13×6×2 mm sheet is assumed as it is smaller which makes it more appropriate for use in a surgically implantable shunt 200.

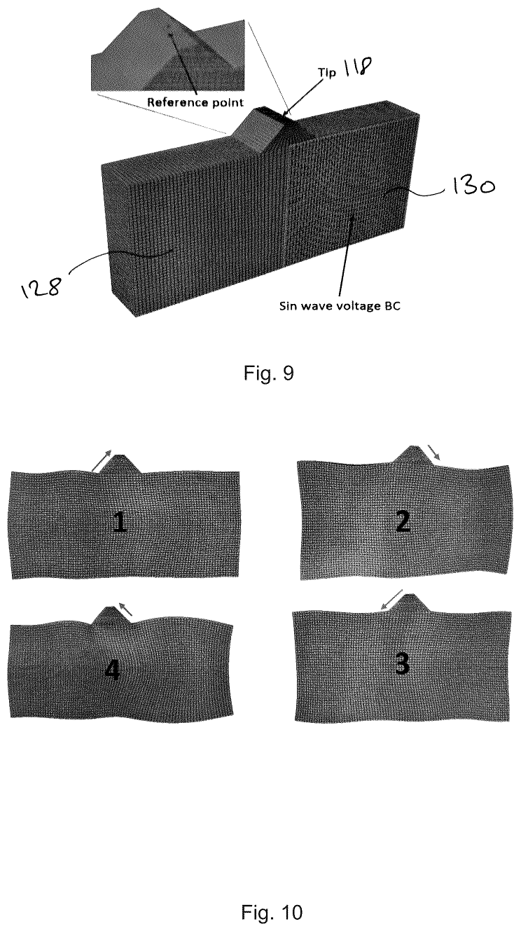

As shown in FIG. 9, to model the gripper (tip) 118, a sin wave voltage of 3.5 V is set as a boundary condition on half of the model face on one side. The frequency of the sin wave is the E(3,1) resonance mode which is at 294.8 kHz. The other side voltage is set to ground across the entire face. The time period on the dynamic simulation is 1.7E-5 s, which is sufficient time for 5 voltage cycles (5*1/f).

Error! Reference source not found. FIG. 10 shows the manner that the gripper 118 is affected by the sin wave voltage. The gripper 118 goes through one cycle movement per voltage cycle. Its changes in position may be classified into 4 points. Stator 114 movement is achieved through this cycle. First, the gripper 118 gains both Y-axis displacement and X-axis displacement. The Y-displacement causes the gripper 118 to clamp on the stator while the X-displacement causes the gripper 118 to drag the stator 114 along with it as a result of friction. Next, negative Y-displacement and X-displacement are gained. This causes the gripper 118 to unclamp and return to its initial position. The four stages in combination are considered a single step of the ultrasonic motor 116.

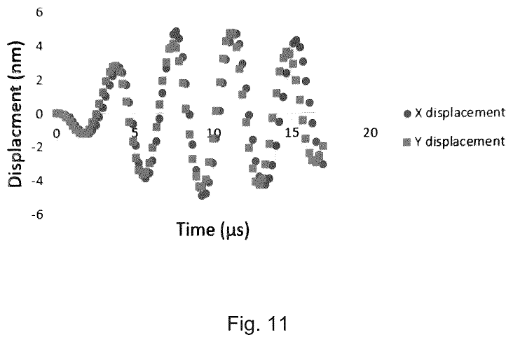

FIG. 11 shows X & Y displacement on the tip according to the modelling referenced point (FIG. 9). The dragging process occurs when the tip is on the positive side of both the X & Y axis. It can be seen from the graph that a single step size is around 4 nm, while the motor 116 has a velocity of 1 mm/sec.

Table 1 presents the number of voltage cycles (i.e., number of motor steps) required to set the opening pressure to each individual pressure point for the above example.

TABLE 1

| | Supine position | | Standing position | |
|---|---|---|---|---|
| | Spring | Piezo | Spring | Piezo |
| | Length | element | Length | element |
| ICP | (mm) | Required cycles | (mm) | Required cycles |
| 20 | 3.33 | 4.22E+04 | 2 | 5.00E+05 |
| 19 | 3.39 | 3.83E+04 | 2.05 | 4.86E+05 |
| 18 | 3.45 | 3.42E+04 | 2.11 | 4.72E+05 |
| 17 | 3.52 | 3.02E+04 | 2.17 | 4.57E+05 |
| 16 | 3.58 | 2.61E+04 | 2.23 | 4.42E+05 |
| 15 | 3.65 | 2.19E+04 | 2.29 | 4.27E+05 |
| 14 | 3.72 | 1.77E+04 | 2.35 | 4.12E+05 |
| 13 | 3.79 | 1.34E+04 | 2.41 | 3.97E+05 |
| 12 | 3.86 | 9.02E+03 | 2.47 | 3.82E+05 |
| 11 | 3.93 | 4.56E+03 | 2.53 | 3.67E+05 |
| 10 | 4 | initial position | 2.6 | 3.51E+05 |

Suitably, it can be seen that the high number of cycles required to achieve a measurable change in length of the mechanical biasing element (spring) 110 allows for extremely fine tuned control of the shunt valve 100. Although 11 ICP points have been discussed as desirable from a design perspective, it can be seen that the presently described shunt valve 100 allows for essentially a continuum of pressure points to be chosen as the threshold pressure for the valve 100 (i.e., the ICP which must be present before the valve opens to allow drain away).

In summary, exemplary embodiments of a compact shunt valve have been described which advantageously does not include many moving parts.

An example valve comprises an inlet in fluid communication with an outlet, a valve closure element, a mechanical biasing element configured to bias the valve closure element towards the inlet, and an electromechanical actuator configured to manipulate the mechanical biasing element. A biasing force applied to the valve closure element by the mechanical biasing element determines an operating pressure of the valve, and manipulating the mechanical biasing element by the electromechanical actuator allows for adjustment of the valve operating pressure by suitably varying the biasing force.

The described embodiments offer a high degree of control over CSF drain (via suitable control of an ultrasonic motor) while beneficially allowing low power usage (due to the motor's low voltage requirement) and an offline locking ability. The combination of the ultrasonic element with the mechanical biasing element means opening pressure values can be set at shorter intervals allowing finer intracranial pressure control. Additionally, the described exemplary embodiments are convenient to manufacture and straightforward to use.

A shunt comprising the example shunt valve may be surgically implanted into or around the cranium—e.g., behind the ear (as in FIG. 2). The valve pressure may be suitably adjusted by application of a suitable voltage to the electromechanical actuator (i.e., to the first or second electrodes, depending on the direction of movement desired), and advantageously the voltage may be controlled post-operation by providing suitable wireless connections to the shunt valve to control applied voltage; appropriately such connections may be accessed without further surgery making the shunt valve adjustable on a day to day basis.

The example embodiments may be manufactured industrially. An industrial application of the example embodiments will be clear from the discussion herein.

Although preferred embodiment(s) of the present invention have been shown and described, it will be appreciated by those skilled in the art that changes may be made without departing from the scope of the invention as defined in the claims.

Attention is directed to all papers and documents which are filed concurrently with or previous to this specification in connection with this application and which are open to public inspection with this specification, and the contents of all such papers and documents are incorporated herein by reference.

All of the features disclosed in this specification, and/or all of the steps of any method or process so disclosed, may be combined in any combination, except combinations where at least some of such features and/or steps are mutually exclusive.

Each feature disclosed in this specification may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification, or to any novel one, or any novel combination, of the steps of any method or process so disclosed.

1. A valve apparatus for a shunt useable in treatment of hydrocephalus, comprising:
an inlet in fluid communication with an outlet;
a valve closure element and a mechanical biasing element configured to bias the valve closure element towards the inlet, wherein a biasing force applied to the valve closure element by the mechanical biasing element determines an operating pressure of the valve, the operating pressure being a minimum fluid pressure able to flow into the valve through the inlet; and
an electromechanical actuator configured to manipulate the mechanical biasing element to adjust the biasing force to vary the operating pressure of the valve.

2. The valve apparatus of claim 1, wherein the electromechanical actuator is self-locking.

3. The valve apparatus of claim 1, wherein the electromechanical actuator comprises a stator driven by an ultrasonic motor.

4. The valve apparatus of claim 3, wherein the ultrasonic motor engages the stator on a surface of the stator running substantially parallel to a direction of the biasing force applied to the valve closure element.

5. The valve apparatus of claim 3, wherein the ultrasonic motor comprises a piezoelectric element.

6. The valve apparatus of claim 5, wherein the ultrasonic motor comprises a protrusion configured to grip the stator, the protrusion being arranged on a first surface of the piezoelectric element orthogonal to a second surface of the piezoelectric element to which voltage is applied.

7. The valve apparatus of claim 6, wherein the protrusion is arranged to straddle a boundary between a first electrode of the piezoelectric element and a second electrode of the piezoelectric element.

8. The valve apparatus of claim 6, wherein the protrusion comprises aluminium oxide.

9. The valve apparatus of claim 5, wherein the piezoelectric element comprises piezoceramic material

10. The valve apparatus of claim 9, wherein the piezoceramic material is PTZ-5H.

11. The valve apparatus of claim 3, wherein the ultrasonic motor operates at voltage between 3 and 5 volts, inclusive.

12. The valve apparatus of claim 1, wherein the mechanical biasing element comprises a substantially linear spring.

13. The valve apparatus of claim 12, wherein the mechanical biasing element comprises Magnesium AZ91E-T6.

14. The valve apparatus of claim 1, wherein the valve closure element is substantially ball shaped.

15. The valve apparatus of claim 14, wherein a diameter of the inlet is substantially 1 millimetre ‘mm’, and a diameter of the ball shaped valve closure element is at least 1.3 mm.

16. The valve apparatus of claim 1, further comprising a valve seat, and wherein the valve closure element is configured to rest in the valve seat.

17. The valve apparatus of claim 16 wherein a shape of the valve seat is configured to correspond to a shape of the valve closure element.

18. A shunt for use in treatment of hydrocephalus comprising the valve apparatus of claim 1.

19. A method of treatment of hydrocephalus comprising surgically implanting the shunt of claim 18.

20. The method of claim 19, further comprising adjusting the operating pressure of the electromechanical actuator post-surgery.
